(19) United States
(12) Patent Application Publication
(10) Pub. No.: US 2023/0181458 A1
(43) Pub. Date: Jun. 15, 2023

(54) MICRO DOSING OF VIRAL VACCINES

(71) Applicant: Jeffrey E. KOZIOL, Madison, WI (US)

(72) Inventor: Jeffrey E. KOZIOL, Madison, WI (US)

(21) Appl. No.: 17/419,994

(22) PCT Filed: Jun. 29, 2021
(86) PCT No.: PCT/US2021/039569

§ 371 Date: Jun. 30, 2021

Related U.S. Application Data
(60) Provisional application No. 63/017,929, filed on Apr. 30, 2020.

(51) Int. Cl. A61K 9/00; A61K 39/12; C12N 7/00; A61K 9/08

(52) U.S. Cl. CPC A61K 9/0048; A61K 39/12; C12N 7/00; A61K 9/08; A61K 2039/525

(57) ABSTRACT

A method of administering a pharmaceutical composition includes the step of administering a sterile unit of vaccine to the ocular mucosa. The vaccine can be made of DNA segments, RNA segments, messenger RNA live virus, or attenuated virus. The unit dosage can be about 20 to 70 microliters. The vaccine is suspended in a solution which closely matches the ocular tear film in terms of pH, osmolality and can be non-preserved and viscous. The pharmaceutical composition is delivered to the surface of the eye in sequential doses over a predetermined period of time to provide a total dosage for providing the vaccine to the patient.

This application claims priority under 35 USC 119 (e) to U.S. Provisional application 63/017,929, filed on Apr. 30, 2020, which is hereby incorporated by reference in its entirety.

### BACKGROUND

### Field of the Disclosure

The present disclosure is directed to delivery method for delivering a vaccine for immunization or for delivery other medication to a patient, and particularly a human patient. The delivery method includes a method of administering micro doses of a vaccine or other medication to the eye and particularly to the ocular mucosa. The delivery method of micro dosages of the vaccine can also be a nasal spray composition.

### Description of the Related Art

Medications, drugs, and vaccines are commonly administered to a patient for therapeutic purposes and for preventing or treating a disease or disorder. The delivery methods are commonly an injection using a needle or cannula that can deliver the vaccine, drug or medication by intradermal or intramuscular delivery. The use of a needle and cannula can be uncomfortable to the patient and cause irritation or inflammation at the delivery site. In addition, delivery by a cannula or needle using a syringe or other delivery device generally requires a trained technician to deliver the vaccine, drug or medication properly. Other known methods of delivery of a vaccine can include oral or nasal delivery routes. The prior vaccines are administered in a single dose or multiple dose depending on the particular vaccine.

While the prior methods are generally suitable for the intended use, there is a continuing need for improved method for delivering medications and vaccine to a patient.

### SUMMARY

A method of delivering and/or administering a pharmaceutical composition to a patient to treat and/or inoculate the patient is described. In one embodiment, the method administers a unit dosage of the composition to the surface of the eye where an active agent in the composition is absorbed into the patient.

A feature of the present disclosure is to provide a method of vaccine administration that can be self-administered or administered by a clinician. The vaccine composition or formulation is able to stimulate both mucosal and systemic immunity in a patient.

In one embodiment, the pharmaceutical composition is a vaccine composition to deliver a small unit dose of the vaccine in amounts of about 20 to 70 microliters. The vaccine composition can be a virus vaccine where a unit dose can contain about 10 to 1000 virus particles.

The pharmaceutical composition can be an ocular composition, such as a solution or suspension of an active agent. The active agent can be a virus vaccine containing about 10 to about 1000 virus particles per unit dosage. In one embodiment, the unit dosage can be delivered to the patient 2-4 times over a period of 2-5 days to provide the desired total dosage to the patient. The total dosage can be about 30 to about 3000 virus particles to the patient over the predetermined period of time. In other embodiments, the virus vaccine can be administered by several doses in a single day spaced apart several hours between doses.

One aspect of the method is for delivering an effective amount of a virus vaccine obtained from live virus, attenuated virus, or DNA strands or segments, RNA segments or messenger RNA live virus to the surface of the eye. The vaccine can be non-preserved and can include viscosity modifying agent to increase the viscosity to a range where the vaccine composition remains on the surface of the eye to a length of time to enable absorption of the vaccine. The virus vaccine composition is administered to the ocular mucosa of the eye in an amount to provide a therapeutically effective amount of the virus vaccine to the patient. The virus vaccine is administered by sequential applications over a predetermined period of time to provide a selected total dosage of the virus vaccine to the patient.

These and other aspects of the method and composition will become apparent from the following detailed description, which disclose various embodiments.

### DETAILED DESCRIPTION

The present disclosure is directed to a composition and method for administering a pharmaceutical composition containing a vaccine, drug or medication to a patient. The method is particularly useful for immunization of a patient through the conjunctival mucosa in the eye of a patient.

The vaccine composition in one embodiment contains an amount of a viral vaccine or viral antigen that can be delivered to the eye of the patient or as a nasal spray. As described herein, a delivery method is provided for administering a therapeutically effective amount of a vaccine, drug or medication to the eye of a patient to treat the patient. The prior vaccines are often non-live vaccines and are delivered intramuscularly. The vaccines typically contain additives, such as aluminum sulfate, formaldehyde, and antibiotics that are not suitable for ocular delivery.

The delivery of the virus vaccine can be by sequential micro-dosing of the vaccine to a patient by several applications to deliver an effective amount of the vaccine or antigen to the patient. The virus vaccine composition contains a micro-dose of the active agent that is administered to the surface of the eye. The unit dosage contains a small dosage that is delivered by sequential applications to provide the intended total dosage to the patient.

The delivery and administration of drugs and medications is of interest to the medical field where the drugs and medications can be delivered quickly and easily. Of particular interest is a rapid and effective delivery route for vaccines that can be used for immunization and to treat or inhibit the spread of a virus and reduce the risk of a pandemic. Examples of virus that have resulted in a pandemic include severe acute respiratory syndrome (SARS) and COVID 19.

The eye includes a mucosa layer that is amenable as a delivery route of a substance to the patient without the need a trained technician to administer an injection to the patient. The method as described herein provides an effect delivery of various substances to a patient by providing a composition, such as a solution or suspension, of an active agent that can be administered to the patient in drop form to the surface of the eye. The composition containing the active agent is easy to administer and does not require special training. The composition for delivery to the eye can be self-administered by the patient.

The eye mucosa is used as route for mucosal vaccine as an entry point to the patient. The eye mucosa, and particularly the conjunctiva has immunological characteristics similar to other mucosal tissue. Therefore, the ocular delivery of a vaccine, drug or medication is selected as the delivery route. The conjunctival epithelium contains a special subpopulation of dendritic APCs known as Langerhans cells, which are capable of both uptake of antigens and priming of naïve T lymphocytes. The dendritic cells serve as the sentinel cell of the immune system of the ocular surface. The conjunctiva contains blood vessels and lymphatic vessels, which facilitate the trafficking of immune cells and antigens to the draining lymph nodes, where the adaptive immune response is generated. This occurs through the recruitment of Treg cells, which return to the ocular surface to modulate and suppress the local immune response.

The composition is generally a solution or suspension containing an active agent to treat the selected disease or disorder. The composition is able to deliver a therapeutically effective amount of the active agent to the patient by administering the composition to the surface of the eye. The composition can be delivered in the form of drops or as an implant that can provide a sustained release of the active agent. In other embodiments, the composition can be delivered as a nasal spray.

The composition in one embodiment contains small amounts of a live or attenuated virus so that very small micro doses are delivered to the eye with each application. The concentration of the vaccine is sufficiently small that multiple micro doses over a selected period of time are needed to provide the intended result. The advantage of the small, micro dose concentration and the delivery in the form of eye drops enable the patient to self-administer the vaccine over a period of hours and/or days depending on the concentration and the rate of uptake by the patient. The micro doses of the composition containing the micro dose of the virus, attenuated virus, or antigen can be delivered by a nasal spray that can be delivered by multiple doses over a selected period of time to deliver an amount of the virus, attenuated virus or antigen to the patient to provide a vaccine of the intended virus.

The present method is particularly directed to a delivery method for administering a vaccine to a patient through the eye mucosa. The vaccine can be a suitable agent that can be prepared in a solution or suspension in an amount such that the vaccine can be delivered to the patient through the eye to provide a therapeutically effective amount of the vaccine to the patient.

One embodiment of the composition in contains a therapeutically effective amount of a sterile virus vaccine or viral antigen. The virus vaccine agent can be an attenuated virus. In one embodiment, the composition contains a live virus in sufficiently low concentrations to be administered by several doses over a selected period of time. In other embodiments, the vaccine can include a live virus or non-live virus in amounts to treat the patient effectively by a single dose to the eye. The vaccine can be isolated from natural sources or synthesized as DNA segments, RNA strands or segments, messenger RNA live virus, or attenuated virus. The composition contains an amount of the vaccine that can be delivered to the ocular mucosa of the eye and absorbed by the patient in time sufficient to treat the patient effectively. The composition can include various additives, excipients, preservatives, and/or stabilizing agents that can be administered to the surface of the eye. In one embodiment, the ocular composition contains no preservatives. The prior injectable vaccines for intramuscular injection typically contain preservatives that, while acceptable for intramuscular injection, are not suitable to use in the eye.

The virus vaccine composition provides a mechanism for the administration of the vaccine that can be self-administered or can be administered by a medical technician. The virus composition is able to stimulate both mucosal and systemic immunity that can be repeated easily.

The composition in one embodiment is in the form of eye drops that contain a vaccine and can be administered by the patient directly to the surface of the eye. The composition can include suitable stabilizing agents to increase the shelf life of the composition. In other embodiments, the composition can be made in as non-preserved artificial tear.

The carrier for the vaccine is an aqueous media having a pH and/or saline concentration that can be administered to the eye with minimal irritation to the eye and without interaction with the active agent. Examples of a suitable carrier can be a commonly used artificial tear formulation. The carrier can also include an additive to adjust or modify the viscosity of the composition to allow the composition to remain on the surface of the eye for a time sufficient to deliver a therapeutically effective amount of the active agent to the eye and to the patient.

Ocular composition includes a wetting agent and/or a viscosity modifying agent to provide a viscosity of about 10 to 300 cps. The ocular composition can have a viscosity of about 50 to 100 cps. In one embodiment, the ocular composition has a viscosity of about 60-90 cps. Suitable thickening agents or viscosity modifying agents can include sodium hyaluronate, trehalose, hydroxypropyl-guar, cellulose, carboxymethylcellulose, polyvinyl alcohol, hyaluronic acid, polymers, saccharides, and polysaccharides. In one embodiment, the ocular composition includes a mixture of carboxymethylcellulose component to provide the desired viscosity for delivery and retention on the surface of the eye to deliver the active agent efficiently. The ocular composition can also include buffers and electrolytes to minimize irritation to the patient.

The ocular composition has a pH suitable for introducing the eye to minimize discomfort to the patient. The ocular composition can have a pH of about pH 6.0 to about pH 9.0. In one embodiment, the ocular composition has a pH of about pH 6.8 to about pH 8.0. In other embodiments, the ocular composition has a pH of about pH 7.4 to about pH 7.8. Normal tear film has a pH 7.6 so that providing a pH close to this range or a neutral pH will minimize discomfort to the patient. The ocular composition has a pH and osmolarity similar to the ocular tear film and can non-preserved and viscous to remain on the surface of the eye for a time sufficient to enable absorption of the vaccine.

The ocular composition can have an osmolarity suitable for applying topically to the eye. In one embodiment, the ocular composition has an osmolarity of about 160 to about 340 mOsm/L. The osmolarity in other embodiments can be 160 to 310 mOsm/L. In another embodiment, the ocular composition has an osmolarity of about 270 to 340 mOsm/L. Normal human tears have an osmolarity of 295 to 300 mOsm/L so that providing an osmolarity with a range that does not result in discomfort to the patient is desirable.

The ocular composition is preferably a stabilized and sterile composition. The composition is typically in the absence of a preservative. The composition can be sterilized by gamma radiation, by steam heat, or by electron beam irradiation. The sterilization step is preferably a process that provides a sterile composition without denaturing the proteins. An RNA vaccine can be sterilized by gamma radiation.

The ocular composition containing the vaccine, such as a live, inactive, or attenuated virus, is present in the composition in lower concentrations than conventionally used in compositions for parental administration. One example of a composition suitable for delivery as eye drops can include vaccine agent in an amount of about up to 50% less than compositions for parental administration. In the embodiments of the vaccine delivery, the composition contains small amounts of the vaccine or virus that can be delivered to the eye without adverse effects.

The eye drop ocular composition is delivered in drop form using a prefilled delivery device directly to the surface of the eye in an amount to treat the patient effectively. The dosage can be modified or adjusted based on the concentration of the active agent, such as a vaccine, and the rate of absorption of the active agent by the eye and introduced into the blood stream of the patient. The eye drops can be micro-dosing of a vaccine. The eye drops can contain as little as one virus particle per eye drop. The eye drop can have a volume of about 20 microliter to about 70 microliter containing one or more virus particles per drop. In one embodiment the unit dosage is about 25 to about 70 microliters. In another embodiment, the unit dosage can be about 25 to 50 microliters. The unit dosage can be administered several times over a period of time to deliver a total dosage of about 75 to 200 microliters.

The drop size can be controlled by the size of the opening in the delivery device or delivery container and the surface tension of the composition. The drops containing the vaccine can be administered at intervals of hours during the same day to provide the intended dosage. The delivery device containing the unit dosage is discarded each day to avoid or minimize contamination. The delivery device is generally a single use eye drop container that is able to deliver a single drop or multiple drops to the eye. In one embodiment, the delivery device is a flexible prefilled container that can be opened by the user and compressed to expel the ocular composition is a drop-wise manner to deliver one or multiple drops to the eye.

The unit dosage containing about 10 to about 1000 virus particles where the unit dosage is about 25 to about 70 microliters. The eye drops can be delivered at very low concentrations of virus particles or antigen in multiple applications over a selected period of time to deliver an effective amount of the vaccine or antigen. The method in one embodiment delivers a unit dosage sequentially over two to three doses for a total dosage of about 30 to about 3000 virus particles.

The concentration of the active agent can be selected such that 1-3 drops of the composition provide a single unit dosage to the patient. The active agent can be included in an amount or concentration so that the composition can be administered by several dosages during a single day or several dosages over several days depending the administration procedure, the concentration of the active agent, and the desired dosage rate. In one embodiment, the concentration of the active agent is sufficiently low so that multiple doses are required over a several applications over a period of time to deliver the vaccine to the patient in an amount effective to treat the patent over a predetermined length of time where the vaccine can be absorbed by the patient to provide the intended vaccination. The composition for delivering the active agent in the form of eye drops directly to the eye can include any suitable virus vaccine agent that can be administered to and through the eye. The virus vaccine can be any suitable vaccine that can be administered to the eye and utilized by the patient.

The ocular composition can be prepared by various methods that will produce a stable and sterile composition. In one embodiment, the composition and delivery device can be prepared by a blow fill seal method as known in the art. The delivery device is prepared from a pharmaceutical grade plastic resin that is heated and extruded into a circular/tubular shape called a parison, which is placed in a two piece mold and cut from the parison. The mold is then transferred to a sterile filling space and the parison expanded by mandrels to form the container shape and volume of the delivery device. The container can have a volume of about 0.1 ml to about 3 ml. The container is then filled with the ocular composition and a secondary top mold seals the container. The process takes place within a strike chamber of the molding machine. The mandrels that are used to inflate and expand the parison can be used to introduce the vaccine into the containers.

The above description of the preferred embodiments is not to be deemed as limiting the disclosure, which is defined by the appended claims. The disclosure is intended to enable the artisan of ordinary skill to practice variants of the disclosure described without departing from the scope of the disclosure. Numerical limitations herein, in the specification and in the claims, are understood to be limited by the modifier “about,” such that minor departures yielding equivalent results is within the scope of the disclosure. Features or dependent claim limitations disclosed in connection with one embodiment or independent claim may be combined in another embodiment or with a different independent claim without departing from the scope of the disclosure.

1. A method of delivering a pharmaceutical composition to a patient, comprising delivering an amount of the pharmaceutical composition containing a virus vaccine to the surface of the eye or nasal mucosa of the patient in an amount to provide a therapeutically effective amount of the active agent to the patient, where the virus vaccine is obtained from the live virus, attenuated vaccine, DNA strand or segments, RNA or messenger RNA.

2. The method of claim 1, wherein said pharmaceutical composition is an aqueous eye drop composition containing a viscosity modifying or thickening agent and where said virus vaccine composition has a viscosity of about 50 to 100 cps.

3. The method of claim 1, wherein the pharmaceutical composition contains a micro dose of the live virus, attenuated virus, or antigen, and where said composition is delivered to the patient by multiple doses over a selected period of time to provide the virus, attenuated virus, or DNA, RNA or messenger RNA in an amount effective to provide a vaccine effect.

4. The method of claim 1, wherein each said dose is an eye drop having a volume of about 25 microliter to about 50 microliter, and where each said drop contains one or more virus particles per drop.

5. The method of claim 1, where said virus vaccine composition has a pH of about 7.4 to pH 7.8.

6. The method of claim 5, wherein said virus vaccine has an osmolarity of 270 to340 mOsm/L.

7. The method of claim 5, where said virus composition includes about 10 to about 1000 virus particles per unit dosage.

8. The method of claim 7, wherein said virus composition is sterilized by gamma radiation.

9. A method of delivering a virus vaccine composition to a patient, comprising sequentially delivering a unit dose amount of the virus vaccine composition to the surface of the eye of the patient in an amount to provide a therapeutically effective amount of the virus vaccine to the patient, where the virus vaccine is obtained from the live virus, attenuated vaccine, or antigen, and the unit dose contains about 10 to 1000 virus particles.

10. The method of claim 9, wherein each said unit dose is an eye drop having a volume of about 25 microliter to about 50 microliter, and where each said drop contains 10 to 1000 virus particles per drop.

11. The method of claim 9, wherein said pharmaceutical composition is an aqueous eye drop composition containing a viscosity modifying or thickening agent and where said virus vaccine composition has a viscosity of about 50 to 100 cps.

12. The method of claim 9, where said virus vaccine composition has a pH of about 7.4 to pH 7.8.

13. The method of claim 12, wherein said virus vaccine has an osmolarity of 270 to 340 mOsm/L.

14. The method of claim 9, wherein a total amount of the virus vaccine delivered to the patient is about 30 to 3000 virus particles.

15. The method of claim 14, wherein the sequential doses are delivered in a single day.

16. The method of claim 15, wherein the sequential doses are delivered over a period of 2-5 days.
